(19) United States
(12) Patent Application Publication
(10) Pub. No.: US 2022/0081606 A1
(43) Pub. Date: Mar. 17, 2022

(54) METHODS OF INHIBITING SCALE WITH ALKYL DIPHENYLOXIDE SULFONATES

(71) Applicant: Kao Corporation, Tokyo (JP)

(72) Inventors: John HOWE, Reidville, NC (US); Mohand MELBOUCI, High Point, NC (US); Andrew HUGHES, Greensboro, NC (US); John THOMPSON, High Point, NC (US)

(73) Assignee: Kao Corporation, Tokyo (JP)

(21) Appl. No.: 17/420,419

(22) PCT Filed: Mar. 25, 2020
(86) PCT No.: PCT/US2020/024670

§ 371 Date: Jul. 2, 2021

Related U.S. Application Data
(60) Provisional application No. 62/829,429, filed on Apr. 4, 2019.

(51) Int. Cl. C09K 8/528; C07C 309/32

(52) U.S. Cl. CPC C09K 8/528; C07C 309/32

(57) ABSTRACT

A method of inhibiting the formation of scale, in particular barium sulfate and strontium sulfate scale, in an oil and gas well servicing fluid, the method involving adding a scale inhibitor composition that includes an alkyl diphenyloxide sulfonate into the oil and gas well servicing fluid. The alkyl diphenyloxide sulfonate is at one of a monoalkyl diphenyloxide monosulfonate, a monoalkyl diphenyloxide disulfonate, a dialkyl diphenyloxide monosulfonate, and a dialkyl diphenyloxide disulfonate.

### BACKGROUND OF THE INVENTION

### Field of the Invention

The present invention relates to methods of inhibiting scale with alkyl diphenyloxide sulfonates.

### Discussion of the Background

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

Aqueous fluids are injected into the earth and/or recovered from the earth during subterranean hydrocarbon recovery processes such as water flooding, hydraulic fracturing (fracking), and tertiary oil recovery. Aqueous fluid which flows back from the subterranean formation as a byproduct along with oil/gas is called “produced water”. Produced water includes one or more of an injected aqueous liquid, connate (native water present in the subterranean formation along with the hydrocarbon), sea water, and minor (e.g., <5 wt. %) amounts of hydrocarbon products (entrained liquids and/or solids).

Produced water is considered to be industrial wastewater, and historically, produced water has been disposed of in large evaporation ponds. However, this has become an increasingly unacceptable disposal method from an environmental perspective. Therefore, produced water (or diluted produced water) is being increasingly reused/recycled by being reinjected back into the subterranean formation as a servicing fluid. For example, in fracking operations, produced water or diluted variants thereof is often used as a base fluid to formulate fracking fluids.

However, produced water is characterized by a high total dissolved solids (TDS) content, sometimes up to 300,000 ppm TDS, and therefore re-injecting produced water as a fracking fluid with such high TDS can interfere with the functioning of certain additives included in the fracking fluid and lead to the formation of scale. Scale is a mineral salt deposit or coating formed on the surface of metal, rock or other material caused by a precipitation phenomenon. Typical scales encountered in oil and gas field environments include calcium carbonate, calcium sulfate, calcium phosphate, barium sulfate, strontium sulfate, iron sulfide, iron oxides, iron carbonate, colloidal silica (polymerized silica particles), as well as the various silicate, phosphate, and/or oxide variants of any of the above. In severe conditions, scale creates a significant restriction, or even a plug, in various process equipment such as production tubing, which can require shut down time for cleaning or equipment replacement.

Of the various types of scale, barium sulfate scale is widely recognized as one of the most difficult to inhibit. In some regions, like the Marcellus shale basin, produced water contains a high barium concertation (around 2,900 ppm barium), and thus barium scale is a significant operations issue.

While phosphonate and polyacrylate-based scale inhibitors are usually acceptable for calcium carbonate and calcium sulfate scale, they are generally ineffective at controlling barium sulfate scales. Further, carboxylate-containing chelating agents require very high dosages and treatment is still often unsuccessful. Currently, the most effective scale inhibitors for barium sulfate scale are based on sulfonated organic polymers. While generally achieving acceptable results, such sulfonated organic polymers are costly, and are thus only used on the most difficult scales/in severe conditions when other scale inhibitors such as phosphonates fail.

### SUMMARY OF THE INVENTION

In view of the forgoing, there is a need for inexpensive scale inhibitor compositions for inhibiting the formation of all different types of scale, particularly barium sulfate scale and/or strontium sulfate scale, and which are effective at low dosages and remain effective under harsh conditions common to oil/gas field environments.

Accordingly, it is one object of the present invention to provide novel methods of inhibiting the formation of scale in an oil and gas well servicing fluid by adding a scale inhibitor composition that includes an alkyl diphenyloxide sulfonate into the oil and gas well servicing fluid.

These and other objects, which will become apparent during the following detailed description, have been achieved by the inventors' discovery that alkyl diphenyloxide sulfonates alone, or in combination with a chelant, such as a phosphate ester or EDTA and/or a dispersant, such as a sulfonated phosphino polycarboxylic co-polymer or a phosphonate, and particularly mixtures of alkyl diphenyloxide sulfonates containing alkyl diphenyloxide monosulfonates, provide a superior antiscalant under harsh conditions common to oil/gas field environments.

Thus, the present invention provides:

(1) A method of inhibiting the formation of scale in an oil and gas well servicing fluid, the method comprising:

adding a scale inhibitor composition comprising an alkyl diphenyloxide sulfonate into the oil and gas well servicing fluid.

(2) The method of (1), wherein the alkyl diphenyloxide sulfonate is at least one compound selected from the group consisting of a monoalkyl diphenyloxide monosulfonate, a monoalkyl diphenyloxide disulfonate, a dialkyl diphenyloxide monosulfonate, and a dialkyl diphenyloxide disulfonate.

(3) The method of (1) or (2), wherein the alkyl diphenyloxide sulfonate is a mixture of a monoalkyl diphenyloxide monosulfonate, a monoalkyl diphenyloxide disulfonate, a dialkyl diphenyloxide monosulfonate, and a dialkyl diphenyloxide disulfonate.

(4) The method of (3), wherein the monoalkyl diphenyloxide monosulfonate and the dialkyl diphenyloxide monosulfonate are present in the mixture in a combined amount of 1 to 15 wt. %, based on a total weight of the mixture.

(5) The method of (3) or (4), wherein the monoalkyl diphenyloxide disulfonate is present in the mixture in an amount of 65 to 93 wt. %, based on a total weight of the mixture.

(6) The method of any one of (3) to (5), wherein the dialkyl diphenyloxide disulfonate is present in the mixture in an amount of 6 to 34 wt. %, based on a total weight of the mixture.

(7) The method of any one of (2) to (6), wherein the monoalkyl diphenyloxide monosulfonate is of formula I, the monoalkyl diphenyloxide disulfonate is of formula II, the dialkyl diphenyloxide monosulfonate is of formula III, and the dialkyl diphenyloxide disulfonate is of formula IV

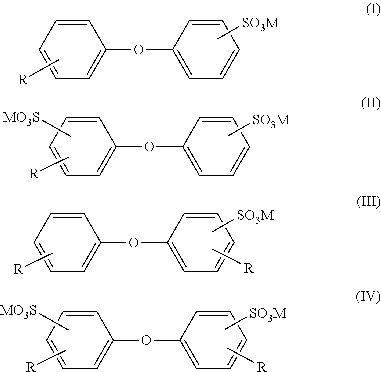

wherein R is an alkyl group with 6 to 22 carbon atoms, and M is selected from H, Na, K, or an ammonium group.

(8) The method of (7), wherein R is an alkyl group with 9 to 14 carbons, and M is Na.

(9) The method of any one of (1) to (8), wherein the alkyl diphenyloxide sulfonate is added into the oil and gas well servicing fluid at a concentration of 100 to 2,000 ppm.

(10) The method of any one of (1) to (9), wherein the scale inhibitor composition further comprises at least one scale inhibitor selected from the group consisting of a phosphate ester, an organic polymer, a phosphonate, and a carboxylate-containing chelating agent.

(11) The method of (10), wherein the scale inhibitor composition is a triblend of the alkyl diphenyloxide sulfonate, the phosphate ester, and a sulfonated phosphino polycarboxylic co-polymer.

(12) The method of (11), wherein a weight ratio of the alkyl diphenyloxide sulfonate to the phosphate ester is 1:3 to 5:1, and a weight ratio of the alkyl diphenyloxide sulfonate to the sulfonated phosphino polycarboxylic co-polymer is 1:3 to 5:1.

(13) The method of (10), wherein the scale inhibitor composition is a triblend of the alkyl diphenyloxide sulfonate, the phosphate ester, and the phosphonate.

(14) The method of (13), wherein a weight ratio of the alkyl diphenyloxide sulfonate to the phosphate ester is 1:3 to 5:1, and a weight ratio of the alkyl diphenyloxide sulfonate to the phosphonate is 1:3 to 5:1.

(15) The method of any one of (11) to (14), wherein the triblend is added into the oil and gas well servicing fluid at a concentration of 1 to 10,000 ppm.

(16) The method of any one of (1) to (15), wherein the oil and gas well servicing fluid is formed from produced water or produced water that has been diluted with fresh water.

(17) The method of any one of (1) to (16), wherein the oil and gas well servicing fluid has a total dissolved solids content of 10,000 to 350,000 ppm.

(18) The method of any one of (1) to (17), wherein the oil and gas well servicing fluid comprises ions of sodium, potassium, magnesium, calcium, strontium, barium, chloride, carbonate, bicarbonate, and sulfate.

(19) The method of any one of (1) to (18), wherein the oil and gas well servicing fluid comprises 100 to 10,000 ppm of Ba²⁺ and the scale comprises barium sulfate scale.

(20) The method of any one of (1) to (19), wherein the oil and gas well servicing fluid comprises 100 to 5,000 ppm of Sr²⁺ and the scale comprises strontium sulfate scale.

(21) The method of any one of (1) to (20), wherein the scale inhibitor composition inhibits the formation of scale at temperatures up to 160° C. in the oil and gas well servicing fluid.

(22) The method of any one of (1) to (21), wherein the scale inhibitor composition inhibits the formation of scale at pressures up to 1,000 psi in the oil and gas well servicing fluid.

### BRIEF DESCRIPTION OF THE DRAWINGS

The foregoing paragraphs have been provided by way of general introduction, and are not intended to limit the scope of the following claims. The described embodiments, together with further advantages, will be best understood by reference to the following detailed description when considered in conjunction with the accompanying drawings, wherein:

FIG. 1 shows the room temperature qualitative visual inspection test results at 1,000 ppm of various scale inhibitor compositions against barium and/or strontium sulfate scale (from left to right: DOWFAX 2A1, PELEX SS-H, PELEX SS-H/DANOX SC-100 (1:1), and blank sample containing no scale inhibitor);

FIG. 2A shows the room temperature qualitative visual inspection test results from various dosages of DOWFAX 2A1 against barium and/or strontium sulfate scale (from left to right: blank sample containing no scale inhibitor, 151 ppm, 307 ppm 585 ppm, 845 ppm, and 1,005 ppm);

FIG. 2B shows a few small scale particles present on the bottom of the 585 ppm vial from FIG. 2A;

FIG. 2C shows no scale particles on the bottom of the 845 ppm vial from FIG. 2A;

FIG. 3 shows a plot of calcium carbonate/sulfate % inhibition as a function of DOWFAX 2A1 concentration according to National Association of Corrosion Engineers (NACE) Standard TM-0374.

FIG. 4 shows a plot of calcium carbonate/sulfate % inhibition as a function of bi-blend and tri-blend scale inhibitor concentration according to National Association of Corrosion Engineers (NACE) Standard TM-0374.

FIG. 5 shows the barium sulfate % inhibition of various scale inhibitors, including triblend, as measured by the ICP analytical method.

### DETAILED DESCRIPTION OF THE INVENTION

In the following description, it is understood that other embodiments may be utilized and structural and operational changes may be made without departure from the scope of the present embodiments disclosed herein.

### Definitions

As used herein, “connate” is native water present in a subterranean formation along with hydrocarbon.

As used herein, “oil and gas well servicing fluid” (or servicing fluid) means water plus any solids, liquids, and/or gasses entrained therein that is injected into a subterranean formation during various drilling operations. Examples of oil and gas well servicing fluids include, but are not limited to, fracking fluids, drilling fluids, completion fluids, and workover fluids.

“Fracking fluid” (or frac fluid) is an injectable fluid used in fracking operations to increase the quantity of hydrocarbons that can be extracted. Fracking fluids contain primarily water, and may contain proppants (e.g., sand) and other desirable chemicals for modifying well production and fluid properties.

“Drilling fluid” is a circulated fluid system that is used to aid the drilling of boreholes, for example, to provide hydrostatic pressure to prevent formation fluids from entering into the wellbore, to keep the drill bit cool and clean during drilling, to carry out drill cuttings, and/or to suspend the drill cuttings while drilling is paused and when the drilling assembly is brought in and out of the hole.

“Completion fluid” is a circulated fluid system that is used to complete/clean an oil or gas well, i.e., to facilitate final operations prior to initiation of production, such as setting screens production liners, packers, downhole valves or shooting perforations into the producing zone. Completion fluids are typically solids-free brines meant to control a well should downhole hardware fail, without damaging the producing formation or completion components.

“Workover fluid” is a circulated fluid system that is used during workover operations, i.e., to repair or stimulate an existing production well for the purpose of restoring, prolonging, and/or enhancing the production of hydrocarbons therefrom.

As used herein, “wastewater” means a water source obtained from storm drains, sedimentation ponds, runoff/outflow, landfills, as well as water sources resulting/obtained from industrial processes such as factories, mills, farms, mines, quarries, industrial drilling operations, oil and gas recovery operations, papermaking processes, food preparation processes, phase separation processes, washing processes, waste treatment plants, toilet processes, power stations, incinerators, spraying and painting, or any other manufacturing or commercial enterprise, which comprises water and one or more compounds or materials derived from such industrial processes, including partially treated water from these sources.

As used herein, “produced water”, a particular type of wastewater, refers to water that flows back from a subterranean formation in a hydrocarbon recovery process and comprises one or more natural formation fluids such as connate, sea water, and hydrocarbon, and optionally any fluid that has been injected into the subterranean formation during various drilling operations.

“Scale” is a mineral salt deposit or coating formed on the surface of metal, rock or other material. Scale is caused by a precipitation due to a chemical reaction with the surface, precipitation caused by chemical reactions, a change in pressure or temperature, or a change in the composition of a solution. Exemplary scales include, but are not limited to, calcium carbonate, calcium sulfate, barium sulfate, strontium sulfate, iron sulfide, iron oxides, iron carbonate, the various silicates and phosphates and oxides, or any of a number of compounds insoluble or slightly soluble in water.

As used herein, “ppm” means parts per million by weight. Except where otherwise noted, all concentrations recited herein are based on weight.

As used herein, “alkoxylated” or “alkoxylate” refers to compounds containing a polyether group (i.e., polyoxyalkylene group) derived from oligomerization or polymerization of one or more alkylene oxides having 2 to 4 carbon atoms, and specifically includes polyoxyethylene (derived from ethylene oxide (E0)), polyoxypropylene (derived from propylene oxide (PO)), and polyoxybutylene (derived from butylene oxide (BO)), as well as mixtures thereof.

The phrase “substantially free”, unless otherwise specified, describes a particular component being present in an amount of less than about 1 wt. %, preferably less than about 0.5 wt. %, more preferably less than about 0.1 wt. %, even more preferably less than about 0.05 wt. %, yet even more preferably 0 wt. %, relative to a total weight of the composition being discussed.

As used herein, the terms “optional” or “optionally” means that the subsequently described event(s) can or cannot occur or the subsequently described component(s) may or may not be present (e.g., 0 wt. %).

The term “alkyl”, as used herein, unless otherwise specified, refers to a straight, branched, or cyclic, aliphatic fragment having 1 to 26, preferably 2 to 24, preferably 3 to 22, preferably 6 to 20, preferably 8 to 18, preferably 10 to 16 carbon atoms. Non-limiting examples include, but are not limited to, methyl, ethyl, propyl, isopropyl, butyl, isobutyl, t-butyl, pentyl, isopentyl, neopentyl, hexyl, isohexyl, 3-methylpentyl, 2,2-dimethylbutyl, 2,3-dimethylbutyl, cyclopropyl, cyclobutyl, cyclopentyl, cyclohexyl, lauryl, myristyl, cetyl, stearyl, and the like, including guerbet-type alkyl groups (e.g., 2-methylpentyl, 2-ethylhexyl, 2-proylheptyl, 2-butyloctyl, 2-pentylnonyl, 2-hexyldecyl, 2-heptylundecyl, 2-octyldodecyl, 2-nonyltridecyl, 2-decyltetradecyl, and 2-undecylpentadecyl), polypropylyl-type alkyl groups (those derived from alkylation of dipropylene, tripropylene, tetrapropylene, pentapropylene, etc.), as well as unsaturated alkenyl and alkynyl variants such as vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, oleyl, linoleyl, and the like.

The term “aryl” refers to a carbocyclic aromatic monocyclic group containing 6 carbon atoms which may be further fused to a second 5- or 6-membered carbocyclic group which may be aromatic, saturated or unsaturated. Exemplary aryl groups include, but are not limited to, phenyl, indanyl, 1-naphthyl, 2-naphthyl and tetrahydronaphthyl.

The term “alkylaryl” refers to aryl groups which are substituted with one or more alkyl groups as defined above, and includes, but are not limited to tolyl, xylyl, ethylphenyl, propylphenyl, and octylphenyl.

The term “arylalkyl” refers to a straight or branched chain alkyl moiety having 1 to 26 carbon atoms that is substituted by an aryl group as defined above, and includes, but is not limited to, benzyl, 2-phenethyl, and 2-phenylpropyl.

As used herein, “inhibit” means prevent, retard, slow, hinder, reverse, remove, lessen, reduce an amount of, or delay an undesirable process or an undesirable composition, or combinations thereof.

As used herein the term “scale inhibitor” refers to a substance(s) that prevents, retards, slows, hinders, or delays the accumulation or buildup of unwanted scale, and/or reverses, cleans, removes, or otherwise reduces/lessens an amount of scale already existing on a surface, for example those surfaces exposed to brine-containing solutions during the production, recovery, transportation, storage and refining of hydrocarbons or various natural gases.

As used herein, “alkyl diphenyloxide sulfonate” is a general term for diphenyloxide compounds that contain at least one alkyl substituent and at least one sulfonate substituent. For example, “alkyl diphenyloxide sulfonate” may refer to, individually or collectively the group of compounds including monoalkyl diphenyloxide monosulfonate (MAMS), monoalkyl diphenyloxide disulfonate (MADS), dialkyl diphenyloxide monosulfonate (DAMS), dialkyl diphenyloxide disulfonate (DADS), etc. “Sulfonate” moieties refer to both sulfonic acid forms as well as sulfonate salt forms (e.g., sodium sulfonate salts).

### Scale Inhibitor Compositions

The present disclosure provides scale inhibitor compositions that include an alkyl diphenyloxide sulfonate, that when added to an oil and gas well servicing fluid provides superior scale inhibition effects against a variety of scale types, particularly against notoriously difficult barium sulfate and/or strontium sulfate scales. The scale inhibitor compositions retain their scale inhibitory effectiveness even when added to oil and gas well servicing fluids having high total dissolved solids content (e.g., up to 350,000 ppm), as well as under the harshest conditions (e.g., temperatures up to 160° C., pressures up to 1,000 psi, etc.) in oil or gas field environments. Further, the alkyl diphenyloxide sulfonate has been found to be surprisingly effective against the most problematic types of scales, such as barium sulfate and/or strontium sulfate scales, at dosages lower than previously thought possible for organic, non-polymeric scale inhibitors.

The scale inhibitor compositions disclosed herein generally include an alkyl diphenyloxide sulfonate, and optionally at least one other scale inhibitor such as a phosphate ester, a sulfonated phosphino polycarboxylic co-polymer or other organic polymer, a phosphonate, and a carboxylate-containing chelating agent. The inventors have also discovered that scale inhibitor compositions that include an alkyl diphenyloxide sulfonate, a phosphate ester, and a sulfonated phosphino polycarboxylic co-polymer or a phosphonate, in particular, are surprisingly effective in combating scale, especially barium sulfate and/or strontium sulfate scales.

### Alkyl Diphenyloxide Sulfonate

Alkyl diphenyloxide sulfonates are compounds containing a diphenyloxide core substituted with at least one alkyl substituent and at least one sulfonate substituent. The alkyl diphenyloxide sulfonate used in the methods described herein may contain the diphenyloxide core substituted with only alkyl substituent(s) and sulfonate substituent(s), or the diphenyloxide core may contain additional types of substitution, for example, halide substituents as disclosed in U.S. Pat. No. 3,634,272—incorporated herein by reference in its entirety.

The alkyl diphenyloxide sulfonates used in the present disclosure may be obtained through any method known to those of ordinary skill in the art (see WO2017196938A1, U.S. Pat. Nos. 6,743,764, 2,990,375, 3,264,242, 3,634,272, 3,945,437, and 5,015,367—each incorporated herein by reference in its entirety, for various alkyl diphenyloxide sulfonates and methods of preparation), typically through a two-step Friedel-Crafts sulfonation process.

In the first step, diphenyloxide may be reacted with an alkylating agent such as an olefin containing 6 to 22 carbon atoms (e.g., tripropylenes, tetrapropylenes, pentapropylenes) or an alkyl halide containing 6 to 22 carbon atoms (e.g., dodecyl bromide), including mixtures of alkylating agents that vary by carbon count and/or linear versus branched constitution, in the presence of a catalyst (e.g., AlCl₃). In some cases, alkylating agents having up to 30 carbon atoms may also be used.

Diphenyloxide may be used in excess and recycled, and the reaction generally yields a mixture of monoalkyl diphenyloxide and dialkyl diphenyloxide, although higher levels of alkylation such as trialkyl diphenyloxide may also be formed by use of high temperatures and high catalyst loadings. The ratio of alkylation (e.g., monoalkylation to dialkylation) can be controlled by adjusting the relative proportions of the reactants. In some embodiments, distillation may be utilized to obtain a fraction containing a mixture of the alkylated diphenyloxides, for example a fraction consisting of or formed predominantly of monoalkyl diphenyloxide and dialkyl diphenyloxide. Alternatively, distillation may be performed so as to separate the alkylated diphenyloxides from one another (and from lower or higher boiling ingredients). For example, a pure fraction of each of the monoalkylated diphenyloxide and the dialkylated diphenyloxide can be obtained, and can be taken forward separately, or recombined at a desirable ratio and subsequently taken forward. In preferred embodiments, a mixture of monoalkylated diphenyloxide and dialkylated diphenyloxide is taken forward.

The alkylated diphenyloxide(s) (e.g., monoalkylated and/or dialkylated diphenyloxide) may then be subsequently reacted with a sulfonating agent, such as chlorosulfonic acid, sulfuric acid, and sulfur trioxide, in an inert solvent (e.g., sulfur dioxide, methylene chloride, carbon tetrachloride, perchloroethylene, etc.). In some embodiments, the sulfonating agent is employed in amounts of at least 1.6, preferably at least 1.7, preferably at least 1.8, preferably at least 1.9, preferably at least 2.0 moles per mole of alkyl diphenyloxide starting material, and up to 3, preferably up to 2.8, preferably up to 2.6, preferably up to 2.5, preferably up to 2.4 moles of sulfonating agent per mole of alkyl diphenyloxide starting material. As a result, the sulfonation reaction generally introduces from 1.5, preferably from 1.6, preferably from 1.7, preferably from 1.8, preferably from 1.9, and up to 2.5, preferably up to 2.4, preferably up to 2.3, preferably up to 2.2, preferably up to 2.1, preferably up to 2 sulfonate moieties per diphenyloxide nucleus. Therefore, the level of sulfonation can be adjusted to improve the yield of monosulfonates, for example, from 5 to 20 wt. % based on a total weight of sulfonated products. Alternatively, the level of sulfonation can be adjusted to favor disulfonation, wherein the reaction product is substantially free of monosulfonates.

Therefore, depending on the purity of the alkyl diphenyloxide(s) subjected to sulfonation, and the sulfonating conditions employed, a variety of products may be optionally obtained in various ratios. In some embodiments, the alkyl diphenyloxide sulfonate used in the methods herein is at least one of a monoalkyl diphenyloxide monosulfonate (MAMS), a monoalkyl diphenyloxide disulfonate (MADS), a dialkyl diphenyloxide monosulfonate (DAMS), and a dialkyl diphenyloxide disulfonate (DADS).

In some embodiments, the alkyl diphenyloxide sulfonate employed is only one of the monoalkyl diphenyloxide monosulfonate (MAMS), the monoalkyl diphenyloxide disulfonate (MADS), the dialkyl diphenyloxide monosulfonate (DAMS), or the dialkyl diphenyloxide disulfonate (DADS).

In some embodiments, the alkyl diphenyloxide sulfonate employed is a mixture of the monoalkyl diphenyloxide monosulfonate (MAMS) and the monoalkyl diphenyloxide disulfonate (MADS), and the scale inhibitor composition is substantially free of dialkyl diphenyloxide monosulfonate (DAMS) and the dialkyl diphenyloxide disulfonate (DADS).

In some embodiments, the alkyl diphenyloxide sulfonate employed is a mixture of the dialkyl diphenyloxide monosulfonate (DAMS), and the dialkyl diphenyloxide disulfonate (DADS), and the scale inhibitor composition is substantially free of the monoalkyl diphenyloxide monosulfonate (MAMS) and the monoalkyl diphenyloxide disulfonate (MADS).

In some embodiments, the alkyl diphenyloxide sulfonate employed is a mixture of dialkyl diphenyloxide disulfonate (DADS) and the monoalkyl diphenyloxide disulfonate (MADS), and the scale inhibitor composition is substantially free of the monoalkyl diphenyloxide monosulfonate (MAMS) and the dialkyl diphenyloxide monosulfonate (DAMS).

In preferred embodiments, the alkyl diphenyloxide sulfonate employed is a mixture of the monoalkyl diphenyloxide monosulfonate (MAMS), the monoalkyl diphenyloxide disulfonate (MADS), the dialkyl diphenyloxide monosulfonate (DAMS), and the dialkyl diphenyloxide disulfonate (DADS). In some embodiments, the monoalkyl diphenyloxide monosulfonate (MAMS) and the dialkyl diphenyloxide monosulfonate (DAMS) are present in the mixture in a combined amount of at least 1 wt. %, preferably at least 2 wt. %, preferably at least 3 wt. %, preferably at least 4 wt. %, preferably at least 5 wt. %, preferably at least 6 wt. %, preferably at least 7 wt. %, preferably at least 8 wt. %, and up to 15 wt. %, preferably up to 14 wt. %, preferably up to 13 wt. %, preferably up to 12 wt. %, preferably up to 11 wt. %, preferably up to 10 wt. %, preferably up to 9 wt. %, based on a total weight of the mixture.

In some embodiments, the monoalkyl diphenyloxide disulfonate (MADS) is present in the mixture in an amount of at least 65 wt. %, preferably at least 66 wt. %, preferably at least 68 wt. %, preferably at least 70 wt. %, preferably at least 72 wt. %, preferably at least 74 wt. %, preferably at least 76 wt. %, and up to 93 wt. %, preferably up to 90 wt. %, preferably up to 88 wt. %, preferably up to 86 wt. %, preferably up to 84 wt. %, preferably up to 83 wt. %, preferably up to 80 wt. %, based on a total weight of the mixture.

In some embodiments, the dialkyl diphenyloxide disulfonate (DADS) is present in the mixture in an amount of at least 6 wt. %, preferably at least 8 wt. %, preferably at least 10 wt. %, preferably at least 12 wt. %, preferably at least 14 wt. %, preferably at least 16 wt. %, preferably at least 18 wt. %, and up to 34 wt. %, preferably up to 32 wt. %, preferably up to 30 wt. %, preferably up to 28 wt. %, preferably up to 26 wt. %, preferably up to 24 wt. %, preferably up to 22 wt. %, preferably up to 20 wt. %, based on a total weight of the mixture.

In preferred embodiments, when a mixture of the monoalkyl diphenyloxide monosulfonate (MAMS), the monoalkyl diphenyloxide disulfonate (MADS), the dialkyl diphenyloxide monosulfonate (DAMS), and the dialkyl diphenyloxide disulfonate (DADS) is employed, the mixture has a net anion to molecule ratio of less than 2.0, preferably less than 1.98, preferably less than 1.96, preferably less than 1.94, preferably less than 1.92, preferably less than 1.9, preferably less than 1.88, preferably less than 1.86, preferably less than 1.85.

In some embodiments, the monoalkyl diphenyloxide monosulfonate (MAMS) is of formula I, the monoalkyl diphenyloxide disulfonate (MADS) is of formula II, the dialkyl diphenyloxide monosulfonate (DAMS) is of formula III, and the dialkyl diphenyloxide disulfonate (DADS) is of formula IV below:

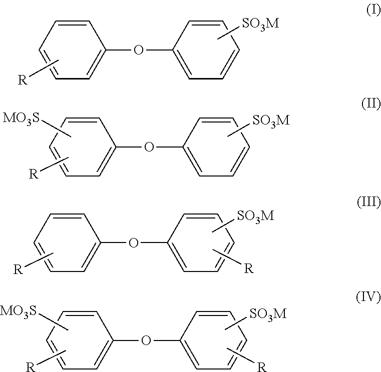

wherein:

R is an alkyl group, preferably a saturated alkyl group, having at least 6, preferably at least 8, preferably at least 9, preferably at least 10, preferably at least 12 carbon atoms, and up to 22, preferably up to 20, preferably up to 18, preferably up to 16, preferably up to 14 carbon atoms; and

M is selected from H, Na, K, or an ammonium group, including mixtures (mixed salts and partially protonated species). In preferred embodiments, M is Na (i.e., the alkyl diphenyloxide sulfonates are in the form of sodium salts).

The R group may a linear alkyl group, a branched alkyl group, or a mixture of linear and branched alkyl groups. In preferred embodiments, R is a fully saturated alkyl group. Representative examples of R groups include, but are not limited to, hexyl, 3-methyl-pentyl, heptyl, octyl, nonyl, decyl, undecyl, dodecyl (lauryl), tridecyl, myristyl, pentadecyl, cetyl, heptadecyl, stearyl, nonadecyl alcohol, arachidyl, heneicosyl, behenyl, isohexyl, 3-methylpentyl, 2,3-dimethylbutyl guerbet-type alkyl groups such as 2-methylpentyl, 2-ethylhexyl, 2-proylheptyl, 2-butyloctyl, 2-pentylnonyl, 2-hexyldecyl, 2-heptylundecyl, 2-octyldodecyl, and 2-nonyltridecyl, and polypropylyl-type alkyl groups such as those derived from alkylation of dipropylene, tripropylene, tetrapropylene, pentapropylene, and higher propylenes, including mixtures thereof.

In some embodiments, for each alkyl diphenyloxide sulfonate present in the scale inhibitor composition (i.e., formula I, II, III, and/or IV), R represents a singular alkyl group (e.g., R is dodecyl). Alternatively, R may represent a mixture of alkyl groups which differ by carbon count, branching, or both, for example, when the diphenyloxide core is alkylated with a mixture of alkylating agents.

In terms of M, when the ammonium group is present, it may have the formula NR¹ₐAr_{b}X_{c}Y_{d}Hₑ, wherein a, b, c, d, and e are individually 0 to 4, and a+b+c+d+e=4; and wherein R¹ is an alkyl group, Ar is an aryl group, X is an alkylaryl group, Y is an arylalkyl group, and H is hydrogen atom. Exemplary ammonium groups include, but are not limited to, ammonium (NH₄⁺), protonated forms of primary, secondary, or tertiary amines (e.g., protonated forms of triethylamine, ethyl amine, butylamine, octyl amine, ethyldiisopropylamine), tetraalkyl ammonium (e.g., tetramethyl ammonium, tetraethyl ammonium, tetrapropyl ammonium, tetrabutyl ammonium, tetrahexyl ammonium, tetraoctyl ammonium, cetyltrimethyl ammonium, distearyl dimethyl ammonium), trialkyl aryl ammonium (e.g., phenyltrimethyl ammonium chloride, phenyltriethyl ammonium), dialkyl diaryl ammonium (e.g., diphenyl dimethyl ammonium, diphenyl diethyl ammonium), trialkyl arylalkyl ammonium (e.g., benzyltrimethyl ammonium, benzyltriethyl ammonium, decyldimethylbenzyl ammonium), and the like.

The concentration of the alkyl diphenyloxide sulfonate in the scale inhibitor compositions employed in the disclosed methods may be from 1 wt. %, preferably from 5 wt. %, preferably from 10 wt. %, preferably from 15 wt. %, preferably from 20 wt. %, preferably from 25 wt. %, preferably from 30 wt. %, preferably from 35 wt. %, preferably from 40 wt. %, preferably from 45 wt. % and up to 80 wt. %, preferably up to 75 wt. %, preferably up to 70 wt. %, preferably up to 65 wt. %, preferably up to 60 wt. %, preferably up to 55 wt. %, preferably up to 50 wt. %, based on a total weight of the scale inhibitor compositions. The balance of the scale inhibitor compositions may be made from water and any optional additional scale inhibitors which will be described hereinafter.

Suitable examples of alkyl diphenyloxide sulfonates that may be utilized in the methods herein, include, but are not limited to, PELEX SS-H (C₉-C₁₄ alkyl, contains up to 1.5 wt. % of alkyl diphenyloxide monosulfonates (MAMS+DAMS)) and PELEX SS-L (C₉-C₁₄ alkyl, contains about 9 wt. % of alkyl diphenyloxide monosulfonates (MAMS+DAMS)), each available from Kao, Inc.; disulfonated products such as DOWFAX products, for example, DOWFAX 2A1 (branched C₁₂ alkyl), DOWFAX C6L (linear C₆ alkyl), DOWFAX 3B2 (linear C₁₀ alkyl), DOWFAX C10L (linear C₁₀ alkyl), DOWFAX 8390 (linear C₁₆ alkyl), DOWFAX 3BO (acid form of DOWFAX 3B2), DOWFAX 2AO (acid from of DOWFAX 2A1), each available from Dow Chemical Company; and disulfonated products such as CALFAX products, for example, CALFAX 10L-45 (linear C₁₀ alkyl), CALFAX 16L-35 (linear C₁₆ alkyl), CALFAX 6LA-70 (linear C₆ alkyl), CALFAX DB-45 (branched C₁₂ alkyl), CALFAX DBA-40 (acid version CALFAX DB-45), and CALFAX DBA-70 (high active, branched C₁₂ alkyl), and CALFAX SS-H, each available from Pilot Chemical Company.

It has been surprisingly found that alkyl diphenyloxide sulfonates provide excellent scale inhibition effects, even at low doses, and are particularly effective at inhibiting barium sulfate scale. This effect is surprising since many alkyl diphenyloxide sulfonates are known and are commonly employed as surfactants, yet such scale inhibition effects have not been identified.

Further, the superior capability of such alkyl diphenyloxide sulfonates to inhibit difficult scales such as barium sulfate could not have been predicted, especially when one considers that organic polymers such as BELLASOL S-50 from BWA Water Additives are regarded as being the most effective for barium sulfate scale inhibition, while non-polymeric organic materials are considered to be unsatisfactory for this purpose.

Even further, it is generally recognized that scale inhibitors require a minimum of two anions per molecule (Kelland, M. A. Production Chemicals for the Oil and Gas Industry, Second edition, CRC Press, 2014, 3.4 Scale inhibition of group II carbonates and sulfates—incorporated herein by reference in its entirety), with more anions per molecule (e.g., 5, 6, 7, etc.) being preferred, for acceptable inhibitory effects to be realized. Therefore, it is also quite unexpected that alkyl diphenyloxide sulfonate mixtures containing a high content (e.g., up to 15% by weight) of alkyl diphenyloxide monosulfonates (e.g., MAMS+DAMS)), that is, mixtures with a net anion per molecule ratio of less than 2.0, have been found to provide exceptional barium sulfate scale inhibition.

### Other Scale Inhibitors

The alkyl diphenyloxide sulfonate may be the only scale inhibitor present in the scale inhibitor compositions. However, in some embodiments, the scale inhibitor compositions may also optionally include one or more other scale inhibitors (in addition to the alkyl diphenyloxide sulfonate). Such additional scale inhibitors may be classified as chelants and/or dispersants, and include, but are not limited to:
phosphate esters; such as those made from blends of polyphosphoric acid (PPA) and/or P₂O₅ with hydroxyamines, e.g., ethanolamine, N-methylethanolamine, N,N-dimethylethanolamine, N-ethylethanolamine, N-propylethanolamine, N-isopropylethanolamine, N,N-diisopropylethanolamine, N-butylethanolamine, diethanolamine, N-methyldiethanolamine, N-ethyldiethanolamine, triethanolamine (TEA), propanolamine (3-Amino-1-propanol), N-methylpropanolamine, N,N-dimethylpropanolamine, dipropanolamine, tripropanolamine, isopropanolamine, N,N-dimethylisopropanolamine, diisopropanolamine, triisopropanolamine, 2-amino-2-methyl-1-propanol, 2-amino-2-ethyl-1,3-propanediol, 4-amino-1-butanol, 2-amino-1-butanol, sec-butanolamine, di-sec-butanolamine, and bishydroxyethylethylene diamine, for example, DANOX SC-100, available from Kao, Inc., which is a 70% by weight active composition of a phosphate ester formed from TEA/PPA; as well as phosphate esters of PPA and/or P₂O₅ with hydroxyamines formed by alkoxylation of a primary or secondary amines, for example, alkoxylates of diethylenetriamine (DETA), triethylenetetraamine (TETA), and/or tetraethylenepentaamine (TEPA), for example as described in U.S. Pat. No. 3,477,956A—incorporated herein by reference in its entirety;
organic polymers, preferably polymers based on non-ionic monomers, anionic monomers, or mixtures thereof; including, but not limited to, polymaleates (e.g., homopolymers of maleic acid (HPMA)), polyacrylates (e.g., acylic acid homopolymer (PAA or HAA), sodium acrylate homopolymer), polymethacrylates, polyacrylamides, polysaccharides including modified polysaccharides (e.g., carboxymethyl inulin), amino acid-based polymers (e.g., polyaspartic acid (PASP) homopolymer and salts thereof), polyethers (e.g., polymers based on polymerization of EO, PO, and/or BO, such as those described in WO2015/195319A1—incorporated herein by reference in its entirety), polymers based on sulfonated monomers such as 2-acrylamido-2-methylpropane sulfonic acid (AMPS), vinylsulfonates (e.g., vinylsulfonic acid and salts thereof), styrene sulfonates, etc.; including modified versions of such polymers as well as blends thereof or copolymers made from two or more types of monomers, for example, maleic acid copolymers, maleic acid terpolymers, sulfonic acid copolymers (SPOCA), sulfonated polyacrylic acid copolymers, modified polyacrylic acids, carboxylate sulfonate copolymers, acrylic acid (AA)/AMPS copolymers, AA/AMPS/non-ionic monomer terpolymers (e.g., AA/AMPS/polyacrylamide terpolymer), carboxylate/sulfonate/maleic acid (MA) terpolymer, AA/MA copolymer (CPMA), sulfonated styrene/MA copolymer, AA/acrylamide copolymer, AMPS/N,N-dimethylacrylamide copolymer, phosphino carboxylic acid (PCA) polymers (e.g., phosphinopolyacrylate), sulfonated phosphino carboxylic acid copolymer (such as BELLASOL S-50 from BWA Water Additives and DREWSPERSE 6980 available from Solenis), partially hydrolyzed polyacrylamide, polyether phosphonic acids (e.g., polyamino polyether methylene phosphonic acid (PAPEMP));
phosphonates; such as aminotris(methylenephosphonic acid) (ATMP), phosphoisobutane tricarboxylic acid (PBTC), 1-hydroxyethylidene diphosphonic acid (HEDP), hexamethylenediamine tetramethylene phosphonic acid (HMDT or HMDTMPA), diethylenetriamine penta(methylenephosphonic acid) (DTPMP), bis(hexamethylene) triamine penta (methylene phosphonic) acid (BHPMP), bis(hexamethylene) triamine pentabis(methylene phosphonic acid) (HMTPMP), pentaethylene hexaamineoctakis (methylene phosphonic acid) (PEHOMP); including aminophosphonates of ethanolamine, ammonia, ethylene diamine, bishydroxyethylene diamine, bisaminoethylether, diethylenetriamine, hexamethylene diamine, hyperhomologues and isomers of hexamethylene diamine, polyamines of ethylene diamine and diethylene tetraamine, diglycolamine and homologues, or similar polyamines or mixtures or combinations thereof
carboxylate-containing chelating agents (non-polymeric) such as ethylene diamine tetraacetic acid (EDTA), diethylene triamine pentaacetic acid (DPTA), hydroxyethylene diamine triacetic acid (HEDTA), ethylene diamine di-ortho-hydroxy-phenyl acetic acid (EDDHA), ethylene diamine di-ortho-hydroxy-para-methyl phenyl acetic acid (EDDHMA), ethylene diamine di-ortho-hydroxy-para-carboxy-phenyl acetic acid (EDDCHA), nitrolotriacetic acid (NTA), thioglycolic acid (TGA), hydroxyacetic acid, citric acid, tartaric acid, as well as the sodium, potassium, and/or ammonium salts thereof;
including mixtures thereof.

When present, the concentration of the one or more other scale inhibitors in the scale inhibitor compositions employed in the disclosed methods may be from 2 wt. %, preferably from 3 wt. %, preferably from 4 wt. %, preferably from 5 wt. %, preferably from 10 wt. %, preferably from 15 wt. %, preferably from 20 wt. %, preferably from 25 wt. %, and up to 50 wt. %, preferably up to 45 wt. %, preferably up to 40 wt. %, preferably up to 35 wt. %, preferably up to 30 wt. %, based on a total weight of the scale inhibitor compositions.

Of course, any other scale inhibitor known to those of ordinary skill in the art may optionally be included in the scale inhibitor compositions for use in the methods herein, so long as those scale inhibitors are compatible with the alkyl diphenyloxide sulfonate.

In some embodiments, the scale inhibitor composition is substantially free of an organic polymer. In some embodiments, the scale inhibitor composition is substantially free of a phosphonate scale inhibitor. In some embodiments, the scale inhibitor composition is substantially free of a carboxylate-containing chelating agent.

In some embodiments, the scale inhibitor composition comprises an alkyl diphenyloxide sulfonate, a phosphate ester, and an organic polymer. In some embodiments, the scale inhibitor composition comprises an alkyl diphenyloxide sulfonate, a phosphate ester, and a sulfonated phosphino polycarboxylic co-polymer. In some embodiments, the scale inhibitor composition comprises an alkyl diphenyloxide sulfonate, a phosphate ester, and a phosphonate. In preferred embodiments, the scale inhibitor composition consists essentially of, or consists of an alkyl diphenyloxide sulfonate, a phosphate ester, and a sulfonated phosphino polycarboxylic co-polymer as scale inhibitor components, along with water, referred to herein as a “triblend”. In preferred embodiments, the scale inhibitor composition consists essentially of, or consists of an alkyl diphenyloxide sulfonate, a phosphate ester, and a phosphonate as scale inhibitor components, along with water, referred to herein as a “triblend”. More preferably the triblend is a mixture of an alkyl diphenyloxide sulfonate, in one or more embodiments, a triethanolamine (TEA)/polyphosphoric acid (PPA) phosphate ester (e.g., DANOX SC-100, available from Kao, Inc.), and a sulfonated phosphino polycarboxylic co-polymer (e.g., DREWSPERSE 6980 available from Solenis). More preferably the triblend is a mixture of an alkyl diphenyloxide sulfonate, in one or more embodiments, a triethanolamine (TEA)/polyphosphoric acid (PPA) phosphate ester (e.g., DANOX SC-100, available from Kao, Inc.), and a phosphonate such as aminotris(methylenephosphonic acid) (e.g., PHOS 2 available from Buckman).

Scale inhibitor compositions that include an alkyl diphenyloxide sulfonate, a phosphate ester, and a sulfonated phosphino polycarboxylic co-polymer (e.g., triblends) may be employed in the methods herein having varying component ratios based on the salt content and properties of the servicing fluid to which they are applied. Typically, such scale inhibitor compositions are used having a weight ratio of the alkyl diphenyloxide sulfonate to the phosphate ester of from 1:3, preferably from 1:2, preferably from 1:1.8, preferably from 1:1.6, preferably from 1:1.4, preferably from 1:1.2, preferably from 1:1, preferably from 1.5:1, preferably from 2:1, and up to 5:1, preferably up to 4:1, preferably up to 4.5:1, preferably up to 4:1, preferably up to 3.5:1, preferably up to 3:1. Also a weight ratio of the alkyl diphenyloxide sulfonate to the sulfonated phosphino polycarboxylic co-polymer typically ranges from 1:3, preferably from 1:2, preferably from 1:1.8, and up to 1:1.6, preferably up to 1:1.4, preferably from 1:1.2, preferably from 1:1, preferably from 1.5:1, preferably from 2:1, and up to 5:1, preferably up to 4:1, preferably up to 4.5:1, preferably up to 4:1, preferably up to 3.5:1, preferably up to 3:1.

Scale inhibitor compositions that include an alkyl diphenyloxide sulfonate, a phosphate ester, and a phosphonate (e.g., triblends) may be employed in the methods herein having varying component ratios based on the salt content and properties of the servicing fluid to which they are applied. Typically, such scale inhibitor compositions are used having a weight ratio of the alkyl diphenyloxide sulfonate to the phosphate ester of from 1:3, preferably from 1:2, preferably from 1:1.8, preferably from 1:1.6, preferably from 1:1.4, preferably from 1:1.2, preferably from 1:1, preferably from 1.5:1, preferably from 2:1, and up to 5:1, preferably up to 4:1, preferably up to 4.5:1, preferably up to 4:1, preferably up to 3.5:1, preferably up to 3:1. Also a weight ratio of the alkyl diphenyloxide sulfonate to the phosphonate typically ranges from 1:3, preferably from 1:2, preferably from 1:1.8, and up to 1:1.6, preferably up to 1:1.4, preferably from 1:1.2, preferably from 1:1, preferably from 1.5:1, preferably from 2:1, and up to 5:1, preferably up to 4:1, preferably up to 4.5:1, preferably up to 4:1, preferably up to 3.5:1, preferably up to 3:1.

### Methods

Petroleum oil and natural gas wells are typically subjected to numerous chemical treatments during their production life to enhance operation and protect the integrity of the asset. The formation of scale and other deposits on/within production equipment, such as tubing, has long been a problem for the oil and gas industry. It is well-known that during the production of oil and gas, brine-containing solutions are injected into, are naturally present within, or flow back from the subterranean formation. A precipitation event may occur during operations, and overtime, scale can buildup on/within various drilling equipment. In severe conditions, scale creates a significant restriction, or even a plug, which can require shut down time for cleaning and/or equipment replacement. Scale formation is problematic for any drilling operation, but is even more troublesome in deep-sea operations where cleaning or replacement of equipment is difficult and costly.

To prevent scale buildup, the industry often turns to scale inhibitors, however, many traditional scale inhibitors are ineffective under harsh conditions, such as in fluids containing a high total dissolved solids (TDS) content, under high temperatures/pressures, and in situations where difficult scales such as barium sulfate scale and strontium sulfate scale are of primary concern. For example, traditional phosphonate and polyacrylate-based scale inhibitors, while usually effective at inhibiting calcium carbonate/sulfate scales, are ineffective at controlling barium sulfate scales in difficult production sites such as the oil fields in Cameroon and the Marcellus Shale basin, even when deployed in extremely high dosages.

The present disclosure thus provides a method for inhibiting the formation of scale in oil and gas field environments. As will become clear, the scale inhibitor compositions herein are surprisingly effective at inhibiting the formation of scale, even the most difficult types of scale (e.g., barium sulfate scale) at low concentration, even under harsh environmental conditions.

The scale inhibitor compositions herein are effective at inhibiting scale in a variety of water sources where scale formation is, or may be, problematic. Such water sources may include salt water (e.g., seawater, coastal aquifers, connate, etc.) and/or wastewater sources, as well as mixtures of salt water and/or wastewater sources with fresh water (e.g., water obtained from streams, rivers, lakes, ground water, aquifers, etc.).

The scale inhibitor compositions may be added to any oil and gas well servicing fluid for use in any drilling operation and/or any oil/gas recovery operation in which subterranean crude oil and/or gas is brought to the surface for transport and/or processing, for example, in secondary recovery operations (e.g., water flooding), enhanced oil recovery, and well-stimulation operations (e.g., hydraulic fracturing). In some embodiments, the scale inhibitor compositions may be added to one or more of a fracking fluid, a drilling fluid, a completion fluid, and a workover fluid. Preferably, the methods herein involve the addition of the scale inhibitor composition, in one or more of its embodiments, into a fracking fluid for use in hydraulic fracturing operations. Fracking is a well stimulation technique in which rock is fractured by a high-pressure injection of fracking fluid into a wellbore to create cracks in the deep-rock formations through which natural gas, petroleum, and brine will flow more freely. Both onshore and offshore drilling operations are contemplated.

### Oil and Gas Well Servicing Fluid Contents

The oil and gas well servicing fluid may be formulated using one or more of fresh water, salt water, and wastewater. In preferred embodiments, the servicing fluid is formed from at least wastewater, more preferably from produced water or produced water that has been diluted with another water source (e.g., fresh water). In some embodiments, the produced water comprises connate, servicing fluid which has been previously introduced into the formation (e.g., water used for water flooding operations), or both.

The produced water may be water that flows back from a subterranean formation in a hydrocarbon recovery process, and is subsequently separated from the bulk hydrocarbon phase but comprises an amount of residual hydrocarbon (typically less than 5 wt. %). Therefore, the method may involve recovering a crude oil/produced water from a subterranean reservoir, separating the crude oil/produced water to provide a produced water and a crude oil, adding the scale inhibitor composition and any other optional chemical or material treatments to the produced water (which may contain residual oil), and using the resulting mixture as an oil and gas well servicing fluid (e.g., a fracking fluid in a hydraulic fracking operation).

As mentioned above, the produced water may be optionally diluted with another water source (make-up water) before, during, and/or after the adding. Depending on the total dissolved solids (TDS) of the produced water, the produced water may be diluted down to 90%, preferably down to 80%, preferably down to 70%, preferably down to 60%, preferably down to 50%, preferably down to 40%, preferably down to 30%, preferably down to 25% by volume with another water source (e.g., fresh water) prior to use, for example, prior to use as a fracking fluid in a fracking operation.

The scale inhibitor compositions are suitable for use in servicing fluids with a total dissolved solids content of up to 350,000 ppm (for example when the servicing fluid is made from produced water) or a TDS content ranging from at least 500 ppm, preferably at least 1,000 ppm, preferably at least 2,000 ppm, preferably at least 3,000 ppm, preferably at least 5,000 ppm, preferably at least 10,000 ppm, preferably at least 15,000 ppm, preferably at least 20,000 ppm, preferably at least 40,000 ppm, preferably at least 50,000 ppm, preferably at least 75,000 ppm, preferably at least 100,000 ppm, preferably at least 125,000 ppm, preferably at least 150,000 ppm, preferably at least 175,000 ppm, preferably at least 200,000 ppm, and up to 350,000 ppm, preferably up to 325,000 ppm, preferably up to 300,000 ppm, preferably up to 275,000 ppm, preferably up to 250,000 ppm, preferably up to 225,000 ppm.

Representative examples of cations which may be optionally present in the oil and gas well servicing fluid (or more specifically the fracking fluid) include, but are not limited to, sodium, potassium, magnesium, calcium, strontium, barium, iron (ferrous and ferric), lead, copper, cobalt, manganese, nickel, zinc, aluminum, chromium, and titanium, as well as mixtures thereof. Representative examples of anions which may be present in the oil and gas well servicing fluid (or more specifically the fracking fluid) include, but are not limited to, chloride, carbonate, bicarbonate, sulfate, bromide, iodide, acetate, hydroxide, sulfide, hydrosulfide, chlorate, fluoride, hypochlorite, nitrate, nitrite, perchlorate, peroxide, phosphate, phosphite, sulfite, hydrogen phosphate, hydrogen sulfate, as well as mixtures thereof.

While the amounts of individual ions present may vary significantly based on the location of the well, the water source used to formulate the servicing fluid, whether or not the water source is diluted, etc., the oil and gas well servicing fluid may generally contain up to 320,000 ppm total of monovalent ions, for example at least 300 ppm, preferably at least 400 ppm, preferably at least 500 ppm, preferably at least 1,000 ppm, preferably at least 2,000 ppm, preferably at least 5,000 ppm, preferably at least 10,000 ppm, preferably at least 15,000 ppm, preferably at least 20,000 ppm, preferably at least 50,000 ppm, preferably at least 100,000 ppm, preferably at least 125,000 ppm, preferably at least 150,000 ppm, preferably at least 175,000 ppm, and up to 320,000 ppm, preferably up to 300,000 ppm, preferably up to 275,000 ppm, preferably up to 250,000 ppm, preferably up to 225,000 ppm, preferably up to 200,000 ppm total of monovalent ions.

In some embodiments, chloride ions may be present in the oil and gas well servicing fluid in amounts of at least 100 ppm, and up to 250,000 ppm, preferably up to 200,000 ppm, preferably up to 175,000 ppm, preferably up to 150,000 ppm, preferably up to 125,000 ppm, preferably up to 100,000 ppm, preferably up to 50,000 ppm, preferably up to 10,000 ppm, preferably up to 5,000 ppm, preferably up to 1,000 ppm, preferably up to 500 ppm. In some embodiments, sodium ions may be present in the oil and gas well servicing fluid in amounts of at least 50 ppm, and up to 50,000 ppm, preferably up to 40,000 ppm, preferably up to 30,000 ppm, preferably up to 20,000 ppm, preferably up to 10,000 ppm, preferably up to 5,000 ppm, preferably up to 1,000 ppm, preferably up to 500 ppm, preferably up to 200 ppm. In some embodiments, potassium ions may be present in the oil and gas well servicing fluid in amounts of at least 5 ppm, and up to 20,000 ppm, preferably up to 15,000 ppm, preferably up to 10,000 ppm, preferably up to 5,000 ppm, preferably up to 1,000 ppm, preferably up to 500 ppm, preferably up to 100 ppm.

The oil and gas well servicing fluid may also generally contain up to 50,000 ppm of multivalent cations (e.g., magnesium ions, calcium ions, ferrous ions, strontium ions, barium ions, lead ions, copper ions, cobalt ions, manganese ions, nickel ions, zinc ions, and/or aluminum ions, etc.), for example at least 50 ppm, preferably at least 75 ppm, preferably at least 100 ppm, preferably at least 150 ppm, preferably at least 200 ppm, preferably at least 500 ppm, preferably at least 1,000 ppm, preferably at least 2,000 ppm, preferably at least 5,000 ppm, and up to 50,000 ppm, preferably up to 40,000 ppm, preferably up to 30,000 ppm, preferably up to 20,000 ppm, preferably up to 10,000 ppm, preferably up to 7,000 ppm, preferably up to 6,000 ppm total of multivalent cations.

In some embodiments, barium ions (Ba²⁺) may be present in the oil and gas well servicing fluid in amounts of at least 100 ppm, preferably at least 200 ppm, preferably at least 400 ppm, preferably at least 600 ppm, preferably at least 800 ppm, preferably at least 1,000 ppm, preferably at least 1,200 ppm, preferably at least 1,400 ppm, preferably at least 1,600 ppm, preferably at least 1,800 ppm, preferably at least 2,000 ppm, preferably at least 2,500 ppm, preferably at least 3,000 ppm, preferably at least 4,000 ppm, and up to 10,000 ppm, preferably up to 9,000 ppm, preferably up to 8,000 ppm, preferably up to 7,000 ppm, preferably up to 6,000 ppm, preferably up to 5,000 ppm, preferably up to 4,800 ppm, preferably up to 4,600 ppm of barium ions.

In some embodiments, strontium ions (Sr²⁺) may be present in the oil and gas well servicing fluid in amounts of at least 50 ppm, preferably at least 100 ppm, preferably at least 200 ppm, preferably at least 400 ppm, preferably at least 600 ppm, preferably at least 800 ppm, preferably at least 1,000 ppm, preferably at least 1,200 ppm, preferably at least 1,400 ppm, preferably at least 1,600 ppm, preferably at least 1,800 ppm, preferably at least 2,000 ppm, and up to 5,000 ppm, preferably up to 4,000 ppm, preferably up to 3,000 ppm, preferably up to 2,500 ppm of strontium ions.

Magnesium ions, for example in amounts up to 2,500 ppm, preferably up to 2,000 ppm, preferably up to 1,500 ppm, preferably up to 1,000 ppm, preferably up to 500 ppm, preferably up to 100 ppm, and/or calcium ions, for example in amounts up to 15,000 ppm, preferably up to 12,000 ppm, preferably up to 10,000 ppm, preferably up to 8,000 ppm, preferably up to 6,000 ppm, preferably up to 4,000 ppm, preferably up to 2,000 ppm, preferably up to 1,000 ppm, preferably up to 500 ppm, may also be present in the servicing fluid.

In one specific example, produced water from the Marcellus shale basin, which is well-known for producing difficult to manage barium sulfate scales, may include at least 24,000 ppm sodium ions, at least 11,000 ppm calcium ions, at least 2,900 ppm barium ions, at least 2,300 ppm strontium ions, and at least 900 ppm magnesium ions.

In some embodiments, the oil and gas well servicing fluid has a pH of at least 1, preferably at least 2, preferably at least 3, preferably at least 4, preferably at least 5, preferably at least 6, preferably at least 7, and up to 14, preferably up to 13, preferably up to 12, preferably up to 11, preferably up to 10, preferably up to 9, preferably up to 8.

In addition to being compatible with the various salts and ionic species provided above, even in water sources having an extremely high TDS content, the scale inhibitor compositions are also compatible with a wide range of components, species, chemistries, materials common to oil and/or gas production. For example, the oil and gas well servicing fluid may be used as a fracking fluid, a drilling fluid, a completion fluid, and/or a workover fluid, and may additionally comprise one or more of oil (e.g., produced petroleum), natural gas, carbon dioxide, hydrogen sulfide, organosulfur (e.g., a mercaptan), hydronium ions, oxygen, etc., as well as one or more of other chemistries/materials known to those of ordinary skill in the art used to effect production or fluid properties during drilling operations such as a proppant, a thickening agent, a hydrate inhibitor, an asphaltene inhibitor, a paraffin inhibitor, an H₂S scavenger, an O₂ scavenger, a CO₂ scavenger, an emulsion modifier (e.g., an emulsifier, a demulsifier, etc.), a foamer, a de-foamer, a buffer, a stabilizing agent, a friction reducing agent, a water clarifier, a breaker, a biocide, a crosslinker, a corrosion inhibitor, a surfactant, a clay swell inhibitor, a metal complexing agent, and a winterizer (e.g., methanol), among many others. Due to the compatibility between the scale inhibitor compositions described herein and such other chemistries/materials, the scale inhibition methods described herein may be performed in conjunction with these known chemical treatments in oil and gas field production, downstream transportation, distribution, and/or refining systems.

### Scale Types

The scale inhibitor compositions of the present disclosure are extremely effective against a variety of scales including, but not limited to, calcium carbonate, calcium sulfate, calcium phosphate, barium sulfate, barium carbonate, strontium sulfate, strontium carbonate, iron sulfide, iron oxides, iron carbonate, colloidal silica (polymerized silica particles), and mixtures thereof, as well as the various silicate, phosphate, and/or oxide variants of any of the above, or any scale formed from any combination of cations and anions listed above, or any of a number of compounds insoluble or slightly soluble in water. In some embodiments, the methods herein are employed for combating mixed scales. In some embodiments, the methods herein are employed for inhibiting scales where phosphonate and/or polyacrylate-based scale inhibitors are expected to be, or are proven to be, ineffective. In preferred embodiments, the methods herein inhibit barium sulfate and/or strontium sulfate scale, preferably barium sulfate scale.

### Dosages and Modes of Adding Scale Inhibitor Compositions

The scale inhibitor compositions and any optional additives/make-up water may be added to the servicing fluid using any addition/dosing/mixing techniques known by those of ordinary skill in the art, including both manual and automatic addition techniques. For example, the addition may be carried out by using inline static mixers, inline mixers with velocity gradient control, inline mechanical mixers with variable speed impellers, inline jet mixers, motorized mixers, batch equipment, and appropriate chemical injection pumps and/or metering systems. The chemical injection pump(s) can be automatically or manually controlled to inject any amount of the scale inhibitor composition suitable for inhibiting scale.

The addition of the scale inhibitor compositions may be performed under static conditions, whereby the servicing fluid (e.g., the fracking fluid) is in a static state during the addition, followed by optional mixing using any of many known large volume mixing devices. Alternatively, the addition may be performed under conditions of flow, whereby the servicing fluid (e.g., the fracking fluid) is placed in a flow state, and the scale inhibitor composition is added or jetted into the flowing servicing fluid. For example, a pumping system can be provided to cycle the servicing fluid through one or more mixing stations where the scale inhibitor composition and any optional additives/make-up water is added as it circulates through the pump.

The scale inhibitor composition may be added directly to the oil and gas well servicing fluid or the scale inhibitor composition may be added to a separate water source to be used as make-up water, and the resulting mixture can be subsequently mixed with a base fluid (e.g., produced water) to form the servicing fluid (e.g., fracking fluid made from diluted produced water). In any of the above applications, the scale inhibitor compositions may be injected continuously and/or in batches.

The effective dosage of the alkyl diphenyloxide sulfonate can be empirically determined by a person of ordinary skill in the art (for example based on the TDS and the ions present) to obtain the desired scale inhibition performance for a particular servicing fluid. In some embodiments, for example when the alkyl diphenyloxide sulfonate is the only scale inhibitor employed, the oil and gas well servicing fluid (e.g., the fracking fluid) is treated with at least 50 ppm, preferably at least 100 ppm, preferably at least 150 ppm, preferably at least 200 ppm, preferably at least 250 ppm, preferably at least 300 ppm, preferably at least 350 ppm, preferably at least 400 ppm, preferably at least 450 ppm, preferably at least 500 ppm, preferably at least 550 ppm, preferably at least 600 ppm, and up to 2,000 ppm, preferably up to 1,800 ppm, preferably up to 1,600 ppm, preferably up to 1,400 ppm, preferably up to 1,200 ppm, preferably up to 1,000 ppm, preferably up to 950 ppm, preferably up to 900 ppm, preferably up to 850 ppm, preferably up to 800 ppm, preferably up to 750 ppm, preferably up to 700 ppm, preferably up to 650 ppm of the alkyl diphenyloxide sulfonate (active), based on a total weight of the oil and gas well servicing fluid. The active amount is based on the amount of alkyl diphenyloxide sulfonate actually dosed, so the servicing fluid is treated with an amount of the scale inhibitor composition sufficient to provide the above ppm concentrations of the alkyl diphenyloxide sulfonate within the servicing fluid.

In preferred embodiments, when the scale comprises, consists essentially of, or consists of barium sulfate and/or strontium sulfate scale, the methods involve adding at least 100 ppm, preferably at least 300 ppm, preferably at least 500 ppm, preferably at least 550 ppm, preferably at least 600 ppm, preferably at least 650 ppm, preferably at least 700 ppm, preferably at least 800 ppm, preferably at least 900 ppm, preferably at least 1,000 ppm of the alkyl diphenyloxide sulfonate (active).

In some embodiments, the minimum induction concentration (MIC) of the alkyl diphenyloxide sulfonate is at least 100 ppm, preferably at least 250 ppm, preferably at least 400 ppm, preferably at least 550 ppm, and up to 650 ppm, preferably up to 600 ppm, preferably up to 580 ppm, preferably up to 560 ppm. As mentioned previously, the effective inhibition of scale, in particular difficult scale varieties like barium sulfate scale, with such low doses of non-polymeric scale inhibitors (e.g., alkyl diphenyloxide sulfonates) is surprising.

Moreover, most scale inhibitors are significantly more effective at controlling calcium scales than barium scales. However, the alkyl diphenyloxide sulfonate has been found herein to inhibit barium sulfate scales as effectively, or more effectively, than calcium scale varieties, with the ratio of the MIC of the alkyl diphenyloxide sulfonate against barium sulfate scale to the MIC of the alkyl diphenyloxide sulfonate against calcium carbonate scale being at least 1:1.2, preferably at least 1:1.1, preferably at least 1:1, preferably at least 1.1:1, preferably at least 1.2:1, and up to 1.5:1.

In embodiments where scale inhibitor compositions which comprises an alkyl diphenyloxide sulfonate, a phosphate ester, and a sulfonated phosphino polycarboxylic co-polymer (e.g., triblends) are employed, the effective dosage of such scale inhibitor compositions (e.g., the combined amount of active alkyl diphenyloxide sulfonate, phosphate ester, and sulfonated phosphino polycarboxylic co-polymer) may be from at least 1 ppm, at least 5 ppm, at least 10 ppm, at least 20 ppm, preferably at least 30 ppm, preferably at least 40 ppm, preferably at least 50 ppm, preferably at least 60 ppm, preferably at least 65 ppm, preferably at least 70 ppm, preferably at least 75 ppm, preferably at least 80 ppm, and up to 10,000 ppm, preferably up to 5,000, preferably up to 1,000 ppm, preferably up to 500 ppm, 200 ppm, preferably up to 180 ppm, preferably up to 160 ppm, preferably up to 140 ppm, preferably up to 120 ppm, preferably up to 100 ppm, preferably up to 90 ppm. In some embodiments, the minimum induction concentration (MIC) of the triblend is at least 50 ppm, preferably at least 55 ppm, preferably at least 60 ppm, preferably at least 65 ppm, and up to 85 ppm, preferably up to 80 ppm, preferably up to 75 ppm.

In embodiments where scale inhibitor compositions which comprises an alkyl diphenyloxide sulfonate, a phosphate ester, and a phosphonate (e.g., triblends) are employed, the effective dosage of such scale inhibitor compositions (e.g., the combined amount of active alkyl diphenyloxide sulfonate, phosphate ester, and phosphonate) may be from at least 1 ppm, at least 5 ppm, at least 10 ppm, at least 20 ppm, preferably at least 30 ppm, preferably at least 40 ppm, preferably at least 50 ppm, preferably at least 60 ppm, preferably at least 65 ppm, preferably at least 70 ppm, preferably at least 75 ppm, preferably at least 80 ppm, and up to 10,000 ppm, preferably up to 5,000, preferably up to 1,000 ppm, preferably up to 500 ppm, 200 ppm, preferably up to 180 ppm, preferably up to 160 ppm, preferably up to 140 ppm, preferably up to 120 ppm, preferably up to 100 ppm, preferably up to 90 ppm. In some embodiments, the minimum induction concentration (MIC) of the triblend is at least 50 ppm, preferably at least 55 ppm, preferably at least 60 ppm, preferably at least 65 ppm, and up to 85 ppm, preferably up to 80 ppm, preferably up to 75 ppm.

### Oil/Gas Field Environment

The method may further involve, after adding the scale inhibitor composition to the servicing fluid, injecting the servicing fluid into a pipe in fluid communication with the subterranean reservoir, optionally under pressure. For example, the scale inhibitor composition may be added to a fracking fluid which is then used to stimulate the well by forming cracks in the deep-rock formations through which natural gas, petroleum, and/or brine will flow more freely.

The scale inhibitor compositions described herein are effective even under harsh conditions which may be encountered during certain drilling operations. In some embodiments, the methods herein are effective at inhibiting scale at temperatures up to 160° C., preferably up to 150° C., preferably up to 140° C., preferably up to 130° C., preferably up to 125° C., preferably up to 120° C., preferably up to 115° C. in oil and gas well servicing fluid.

In some embodiments, the methods herein are effective at inhibiting scale at pressures up to 1,000 psi, preferably up to 800 psi, preferably up to 600 psi, preferably up to 400 psi, preferably up to 200 psi, preferably up to 100 psi, preferably up to 80 psi, preferably up to 60 psi, preferably up to 40 psi, preferably up to 35 psi, preferably up to 30 psi, preferably up to 25 psi, preferably up to 20 psi in oil and gas well servicing fluid.

The methods herein may be effective at inhibiting scale buildup on a variety of drilling machinery/equipment/structures, including, but not limited to, gas lines, pipes and/or pipelines, channels, troughs, launders, chutes, ducts, valves, gauges, stopcocks, flowmeters, spools, fittings (e.g., such as those that make up the well Christmas tree), tanks (e.g., treating tanks, storage tanks, etc.), coils of heat exchangers, electrical submersible pumps and pump parts (e.g., parts of beam pumps, sucker rods, etc.), screens, and the like, as well as any other surface known to those of ordinary skill in the art that may be in contact with brine-containing fluids encountered during drilling operations.

The methods herein may be effective at inhibiting scale buildup on a variety of different materials, including, but not limited to, metals such as carbon steels (e.g., mild steels, high-tensile steels, higher-carbon steels), high alloy steels (e.g., chrome steels, ferritic alloy steels, austenitic stainless steels, precipitation-hardened stainless steels high nickel content steels), galvanized steel, aluminum, aluminum alloys, copper, copper nickel alloys, copper zinc alloys, brass, ferritic alloy steels; fiberglass and fiberglass composites; plastics; rock; and/or concrete.

### Scale Inhibition Measurements

In the present disclosure, scale inhibitor compositions which are considered to be “effective” against scale are those which achieve at least one of the following:

1) a % scale inhibition of at least 50%, as determined by quantitative titration-based static tests or Inductively Coupled Plasma-Atomic Emission Spectroscopy (ICP-AES)-based laboratory tests, for example, according to National Association of Corrosion Engineers (NACE) Standard TM-0374 (“Laboratory Screening Tests to Determine the Ability of Scale Inhibitors to Prevent the Precipitation of Calcium Sulfate and Calcium Carbonate from Solution for Oil and Gas Production Systems”—incorporated herein by reference in its entirety) or NACE standard TM-0197 (“Laboratory Screening Test to Determine the Ability of Scale Inhibitors to Prevent the Precipitation of Barium Sulfate or Strontium Sulfate, or Both, from Solution for Oil and Gas Production Systems”—incorporated herein by reference in its entirety); and/or

2) a rating of “clear(+)” or “clear(−)” based on the qualitative visual inspection method described in the examples below.

Therefore, the minimum induction concentration (MIC) of the scale inhibitor compositions of the present disclosure are those dosages which achieve a % scale inhibition of at least 50%, or those dosages where the rating first turns from “cloudy” to either “clear(+)” or “clear(−)”.

Accordingly, in preferred embodiments, the methods utilize a dosage of the scale inhibitor composition which achieves a % scale inhibition of at least 50%, preferably at least 60%, preferably at least 70%, preferably at least 80%, preferably at least 90%, preferably at least 95%, preferably at least 96%, preferably at least 97%, preferably at least 98%, preferably at least 99%, and/or a rating of “clear(+)” or “clear(−)”, preferably “clear(−)”.

### Cleaning Methods

Also contemplated herein are methods of removing, lessening, reducing, shrinking, cleaning, and/or eradicating an existing scale deposit from a surface (e.g., surface of drilling equipment/structures) with the scale inhibitor compositions of the present disclosure, in one or more of their embodiments. In such methods, the scale inhibitor composition may be poured over, pumped over, sprayed, dropped, used as a soak for, or otherwise brought into contact with, a surface having a scale deposit. The scale type may be any of those mentioned previously, preferably difficult scales such as barium sulfate and/or strontium sulfate scales.

The surface having the scale deposit may be contacted with the scale inhibitor composition for any amount of time appropriate to lessen, reduce, shrink, or remove the scale deposit, typically for at least 1 minute, preferably at least 5 minutes, preferably at least 10 minutes, preferably at least 30 minutes, preferably at least 1 hour, preferably at least 2 hours, preferably at least 5 hours, preferably at least 10 hours, preferably at least 12 hours, preferably at least 18 hours, and up to 30 days, preferably up to 20 days, preferably up to 10 days, preferably up to 5 days, preferably up to 1 day.

In some embodiments, the alkyl diphenyloxide sulfonate and any other optional scale inhibitor (e.g.; the phosphate ester and the sulfonated phosphino polycarboxylic co-polymer or the phosphate ester and the phosphonate to make the triblend) may be added to any water source, preferably a fresh water source, to form the scale inhibitor composition for use as a cleaning solution. The concentration of the alkyl diphenyloxide sulfonate, and any other optional component may be as described previously, although more concentrated scale inhibitor compositions are also contemplated.

The surface having the scale deposit may be in contact with a substantially stationary body of the scale inhibitor composition (e.g., soaking methods). Alternatively, the surface having the scale deposit may be brought into contact with the scale inhibitor composition that is in a flowing state, for example, where a stream of the scale inhibitor composition is jetted/impinged onto the surface having the scale deposit, or where a stream of the scale inhibitor composition is flowed or passed over the surface having the scale deposit. For example, when the surface having the scale deposit is an inside surface of a tube/pipe, the scale inhibitor composition may be flowed or passed through the tube/pipe in a direction substantially parallel to the longitudinal axis of the tube/pipe. The stream of the scale inhibitor composition may be flowed or passed over the surface having the scale deposit at an average fluid velocity of at least 0.1 meters per minute (m/min), preferably at least 0.5 m/min, preferably at least 1 m/min, preferably at least 5 m/min, preferably at least 10 m/min, preferably at least 30 m/min, preferably at least 50 m/min, and up to 500 m/min, preferably up to 400 m/min, preferably up to 300 m/min, preferably up to 200 m/min, preferably up to 100 m/min, preferably up to 75 m/min.

The examples below are intended to further illustrate protocols for preparing and testing the scale inhibitor compositions and are not intended to limit the scope of the claims.

### EXAMPLES

### Scale Inhibitor Compositions

Several example scale inhibitor compositions are given below. DOWFAX 2A1 is commercially available from Dow Chemical Company. PELEX SS-H, PELEX SS-L, and DANOX SC-100 are commercially available from Kao. DREWSPERSE 6980 is commercially available from Solenis. PHOS 2 is commercially available from Buckman.

### Example 1 (DOWFAX 2A1)

DOWFAX 2A1 (branched C₁₂ alkyl diphenyloxide disulfonate) is used as is (45% active solution by weight).

### Example 2 (PELEX S S-H)

PELEX SS-H (C₉-C₁₄ alkyl, contains up to 1.5 wt. % of alkyl diphenyloxide monosulfonates (MAMS+DAMS)) is used as is (50% active solution by weight).

### Example 3 (PELEX SS-L)

PELEX SS-L (C₉-C₁₄ alkyl, contains about 9 wt. % of alkyl diphenyloxide monosulfonates (MAMS+DAMS)) is used as is (50% active solution by weight).

### Example 4 (PELEX SS-H: DANOX SC-100=1:1)

A diblend of PELEX SS-H from Example 2 and DANOX SC-100 in a 1:1 ratio based on % actives.

### Example 5 (PELEX SS-H: DANOX SC-100: DREWSPERSE 6980=2:1:1)

A triblend of PELEX SS-H from Example 2, DANOX SC-100, and DREWSPERSE 6980 in a 2:1:1 ratio based on % actives.

### Example 6 (PELEX SS-L: DANOX SC-100: DREWSPERSE 6980=2:1:1)

A triblend of PELEX SS-L from Example 3, DANOX SC-100, and DREWSPERSE 6980 in a 2:1:1 ratio based on % actives.

### Example 7 (PELEX SS-L: DANOX SC-100: PHOS 2=2:1:1)

A triblend of PELEX SS-L from Example 3, DANOX SC-100, and PHOS 2 in a 2:1:1 ratio based on % actives.

### Calcium Scale Inhibition Testing Procedures

### Quantitative Titration-Based Static Laboratory Testing Method:

The National Association of Corrosion Engineers (NACE) Standard TM-0374 (“Laboratory Screening Tests to Determine the Ability of Scale Inhibitors to Prevent the Precipitation of Calcium Sulfate and Calcium Carbonate from Solution for Oil and Gas Production Systems”) was used to determine the ability of scale inhibitor compositions to prevent the precipitation of calcium sulfate and calcium carbonate from solution, with the results being presented in terms of percent inhibition.

Brines were prepared according to NACE Standard TM 0374 procedures (Brine-1 composition in Table 1).

TABLE 1
Brine-1 composition for NACE TM-0374 evaluation

| | CaCl₂•2H₂O | MgCl₂•6H₂O | NaCl | NaHCO₃ |
|---|---|---|---|---|
| Calcium brine (g/L) | 12.15 | 3.69 | 33.00 | — |
| Carbonate brine (g/L) | — | — | 33.00 | 7.36 |

Briefly, the Ca²⁺ concentration of the blank solution was determined before and after precipitation. The precipitation procedure was conducted by immersing the blank test cell to 75% of its length in a water bath at 71±1° C. (160±2° F.) for a 24 hour residence time. After the 24-hour exposure, the test cell was removed from the water bath carefully to avoid any agitation. The test cell was allowed to cool to 25±5° C. (77±9° F.) for a time not to exceed two hours.

The percentage inhibition was calculated using the following relationship:

$$\% inhibition = ( \frac{{C}_{a} - {C}_{b}}{{C}_{c} - {C}_{b}} ) \times 100$$

where Cₐ is the concentrations of calcium ions (Ca²⁺) in the treated sample after precipitation, C_{b} is the concentrations of calcium ions (Ca²⁺) in the blank after precipitation, C_{c} is the concentrations of calcium ions (Ca²⁺) in the blank before precipitation.

The results of % inhibition were then plotted as a function of concentration of the scale inhibitor (active) in units of mg/L (ppm).

### Barium and/or Strontium Scale Inhibition Testing Procedures

### Qualitative Visual Inspection Methods:

(room temperature)—Test brines (Brine-2 in Table 2) were prepared according to the following procedure. An anionic brine solution containing NaHCO₃, Na₂CO₃, and Na₂SO₄ was prepared. A cationic brine solution containing NaCl, KCl, MgCl₂, CaCl₂, SrCl₂, and BaCl₂ was prepared. For preparing the blank sample, equal volumes of the anionic brine solution and the cationic brine solution were mixed in a vial at 25±5° C. (77±9° F.). For preparing the treated samples, the scale inhibitor compositions were added (in different ppm concentrations in terms of active scale inhibitors) to the anionic brine prior to mixing with the cationic brine solution at 25±5° C. (77±9° F.).

TABLE 2
Brine-2 composition for barium sulfate scale inhibitor

| | | | MgCl_{2.} | CaCl_{2.} | SrCl_{2.} | BaC1_{2.} | | | |
|---|---|---|---|---|---|---|---|---|---|
| | NaCl | KCl | 6H₂O | 2H₂O | 6H₂O | 2H₂O | NaHCO₃ | Na₂CO₃ | Na₂SO₄ |
| Cation | 64.26 | 8.4 | 17.44 | 92.12 | 6.09 | 6.82 | — | — | — |
| brine | | | | | | | | | |
| (g/L) | | | | | | | | | |
| Anion | — | — | — | — | — | — | 0.14 | 0.03 | 0.72 |
| brine | | | | | | | | | |
| (g/L) | | | | | | | | | |

For all prepared test brines (blank samples and treated samples prepared from Brine-2), the total dissolved solid (TDS) content was 80,350 ppm, with a barium ion (Ba²⁺) concentration of 1,917 ppm and a strontium ion (Sr²⁺) concentration of 1,000 ppm.

After mixing, the blank sample and the treated solutions were visually inspected for the formation of a precipitate (typically a white precipitate) within 60 seconds of mixing. Tests resulting in cloudy suspensions (significant scale formation occurring) were given a “cloudy” rating. Tests resulting in clear solutions with a few small scale particles settled on the bottom of the jar were given a “clear(+)” rating, (“+” indicating the presence of a few small scale particles). Tests resulting in clear solutions with no signs of settled scale particles on the bottom of the jar were given a “clear(−)” rating (“−” indicating absence of settled scale particles).

(high temperature)—The scale inhibitor compositions were also evaluated under high temperature/high pressure conditions to simulate down well conditions. The vials of treated samples prepared above were subjected to a temperature of 122° C. and a pressure of 25 to 30 psi for 72 hours in a Parr reactor, then allowed to cool to 25±5° C. (77±9° F.) for a time not to exceed two hours. After which, the treated samples were visually inspected for the formation of a precipitate and rated according to the “cloudy”, “clear(+)”, or “clear(−)” rating system described above.

### Quantitative Titration-Based Static Laboratory Testing Method:

The NACE standard TM-0197 (“Laboratory Screening Test to Determine the Ability of Scale Inhibitors to Prevent the Precipitation of Barium Sulfate or Strontium Sulfate, or Both, from Solution for Oil and Gas Production Systems”) is used to determine the ability of scale inhibitor compositions to prevent the precipitation of barium sulfate scale and/or strontium sulfate scale from solution, with the results being presented in terms of percent inhibition.

Similar protocols to the NACE Standard TM-0374 are used, except for the test brines are prepared according to NACE standard TM-0197, and in the percent inhibition equation, Cₐ is the concentrations of barium ions (Ba²⁺) or strontium ions (Sr²⁺) in the tested sample after precipitation, C_{b} is the concentrations of barium ions (Ba²⁺) or strontium ions (Sr²⁺) in the blank after precipitation, C_{c} is the concentrations of barium ions (Ba²⁺) or strontium ions (Sr²⁺) in the blank before precipitation.

### Quantitative Inductively Coupled Plasma-Atomic Emission Spectroscopy (ICP-AES)-Based Laboratory Testing Methods:

The blank sample and treated samples (prepared from Brine-2, Table 2) from the qualitative visual inspection methods above were filtered to remove any precipitate, and the filtrate was subjected to ICP-AES measurements to determine the barium and/or strontium ion concentrations. The barium and/or strontium ion concentrations were plotted as a function of the scale inhibitor (active, ppm), with the higher barium and/or strontium readings being indicative of less precipitation (less scale formation), and thus better % inhibition.

The percentage inhibition was calculated using the following relationship:

$$\% inhibition = ( \frac{{C}_{a} - {C}_{b}}{{C}_{c} - {C}_{b}} ) \times 100$$

where Cₐ is the measured [Ba²⁺] after treatment, C_{b} is the blank [Ba²⁺] after precipitation, C, is the blank [Ba²⁺] initial input before precipitation.

### Scale Inhibition Testing Results

Scale inhibitor compositions were tested for inhibition of barium and/or strontium sulfate scale according to the room temperature qualitative visual inspection method as described above (performed by mixing at 25±5° C. (77±9° F.), with inspection taking place within 60 seconds after mixing), unless otherwise noted.

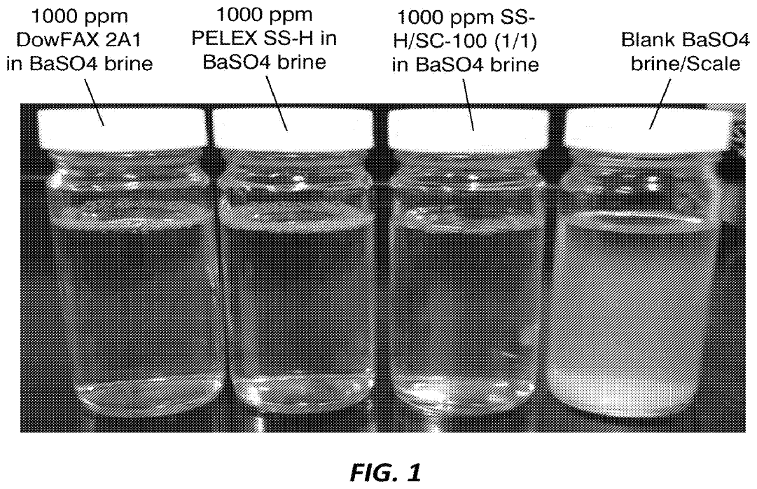

At dosages of 1,000 ppm (active) all scale inhibitor compositions containing alkyl diphenyloxide sulfonate scale inhibitors were effective against barium sulfate and/or strontium sulfate scale, with clear(−) ratings being obtained. The results are presented in Table 3 below and FIG. 1.

TABLE 3
Scale inhibitor compositions against barium
sulfate and/or strontium sulfate scale

| | Scale inhibitor | Scale inhibitor | |
|---|---|---|---|
| | composition | concentration | Qualitative |
| Example | description | (active, ppm) | Rating |
| Example 1 | DOWFAX 2A1 | 1,000 | clear(−) |
| Example 2 | PELEX SS-H | 1,000 | clear(−) |
| Example 4 | PELEX SS-H:DANOX | 1,000^{a)} | clear(−) |
| | SC-100 (1:1) | | |
| Blank | — | — | cloudy |
| Example 1 | DOWFAX 2A1 | 1,000 | clear(−)^{b)} |

^{a)}Total ppm of actives (500 ppm each)
^{b)}Test performed at 122° C. and a pressure of 25 to 30 psi for 72 hours in a Parr reactor

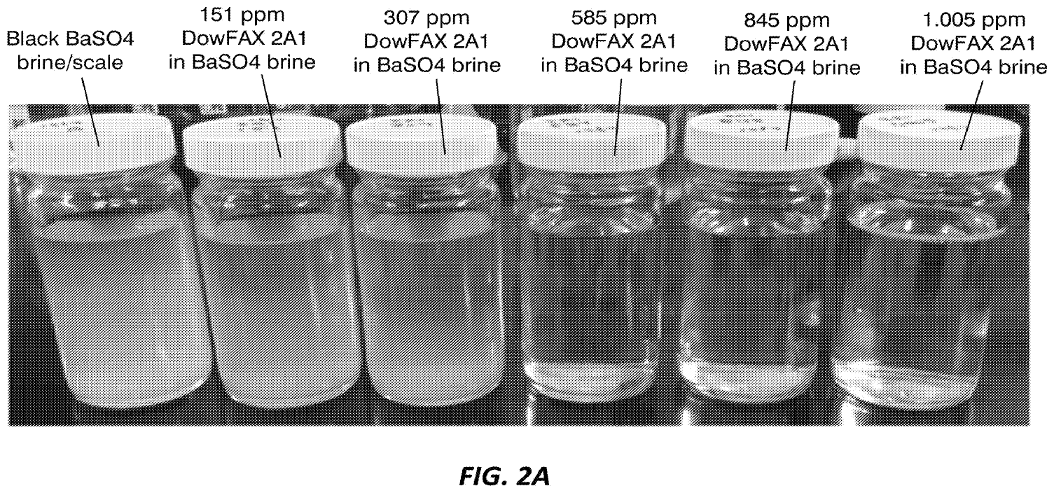

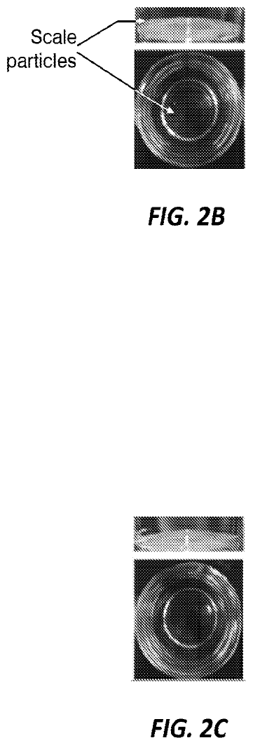

Several scale inhibitor compositions were also tested at various dosages for inhibition of barium and/or strontium sulfate scale according to the room temperature qualitative visual inspection method described above. The results are presented in Table 4 below and FIGS. 2A-2C.

As can be seen from these results, as little as 585 ppm of DOWFAX 2A1 provided effective inhibition of barium and/or strontium sulfate, with only a few scale particles settling at the bottom of the vial (see FIG. 2B). Therefore, when alkyl diphenyloxide sulfonate is the only scale inhibitor added, the minimum induction concentration (MIC) is around 500-600 ppm. Increasing the dosage of DOWFAX 2A1 to 845 ppm resulted in completely clear solutions with no scale precipitates formed (see FIG. 2C).

These results also demonstrate the effectiveness of a triblend of PELEX SS-H: DANOX SC-100: DREWSPERSE 6980 (Example 5, Table 4) at low dosages, where adding only 75 ppm total of the triblend (based on actives) resulted in completely clear solutions with no barium/strontium scale precipitates formed. Because the minimum induction concentration (MIC) when the alkyl diphenyloxide sulfonate was used alone was about 500-600 ppm, one would assume that the MIC of the triblend would similarly be in the 500-600 ppm (total actives) range. However, the MIC of the triblend was found to be nearly an order of magnitude lower, revealing a synergistic effect between the components of the triblend (i.e., alkyl diphenyloxide sulfonate, the phosphate ester, and the sulfonated phosphino polycarboxylic co-polymer).

TABLE 4
Concentration of Scale inhibitor compositions against
barium sulfate and/or strontium sulfate scale

| | Scale inhibitor | Scale inhibitor | |
|---|---|---|---|
| | composition | concentration | Qualitative |
| Example | description | (active, ppm) | Rating |
| Blank | — | — | cloudy |
| Example 1 | DOWFAX 2A1 | 151 | cloudy |
| Example 1 | DOWFAX 2A1 | 307 | cloudy |
| Example 1 | DOWFAX 2A1 | 585 | clear(+) |
| Example 1 | DOWFAX 2A1 | 845 | clear(−) |
| Example 1 | DOWFAX 2A1 | 1,005 | clear(−) |
| Example 5 | PELEX SS-H:DANOX | 75 ^{a)} | clear(−) |
| | SC-100:DREWSPERSE | | |
| | 6980 (2:1:1) | | |

^{a)} total ppm of actives

Several scale inhibitor compositions were also tested at various dosages for inhibition of barium and/or strontium sulfate scale according to the quantitative Inductively Coupled Plasma-Atomic Emission Spectroscopy (ICP-AES)-based laboratory testing method, and the results are presented in Table 5 below.

TABLE 5
Barium sulfate scale inhibition testing per ICP analytical method

| | | | Ba²⁺ Concentration, | | % Ba²⁺ Scale | |
|---|---|---|---|---|---|---|
| | | | ppm (Measured) | | Inhibition (Calculated) | |
| Scale Inhibitor | | | 100 ppm | 500 ppm | 100 ppm | 500 ppm |
| Blank | Initial Ba²⁺ input into | 1917 | — | — | — | — |
| | brine (calculated), ppm | | | | | |
| | Ba²⁺ measured after | 1650 | | | | |
| | scaling, ppm | | | | | |

| | | | | |
|---|---|---|---|---|
| Pelex SS-L (Example 3) | 1720 | 2000 | 26.2 | 100 |
| Pelex SS-H (Example 2) | 1620 | 1670 | 0 | 7.5 |
| Pelex SS-L/Drewsperse 6980/Danox | 1580 | 1780 | 6.0 | 67.0 |
| SC-100 Tri-Blend (2/1/1) (Example 6) | | | | |
| Pelex SS-L/PHOS 2/Danox SC-100 | 1770 | 1800 | 63.6 | 72.7 |
| Tri-Blend (2/1/1) (Example 7) | | | | |

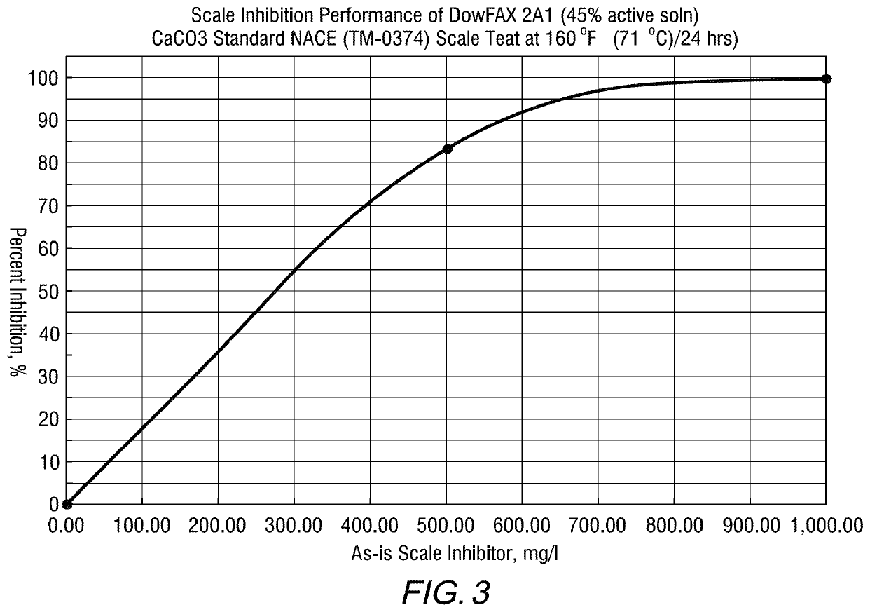

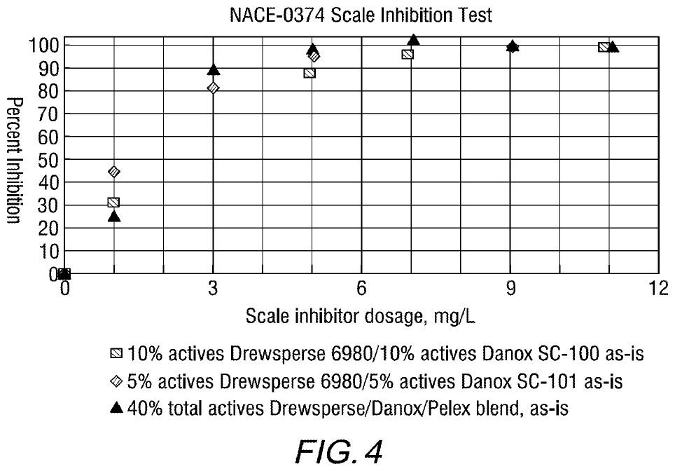

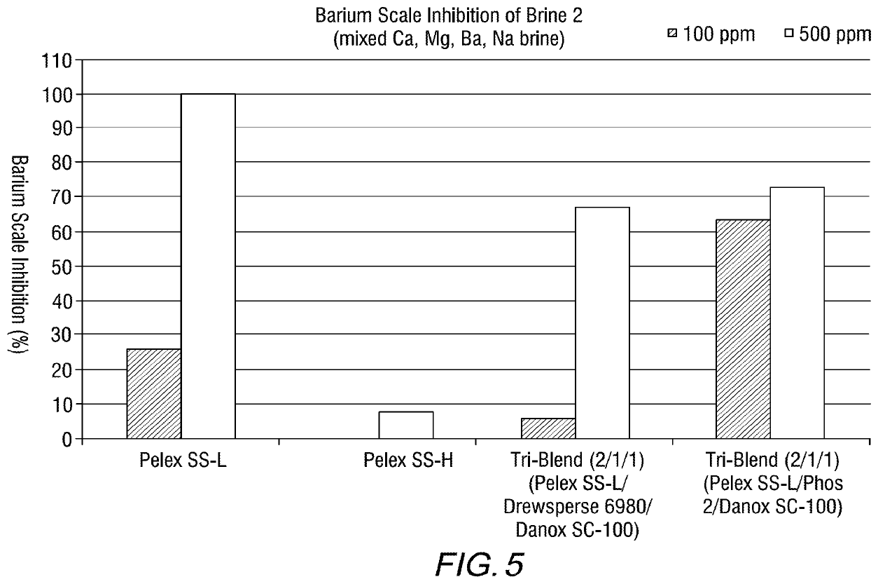

The scale inhibitor composition of Example 1 (DOWFAX 2A1) was also tested at various concentrations as an inhibitor for calcium carbonate and sulfate scales according to the quantitative titration-based static laboratory testing method (NACE Standard TM-0374) described above. The % inhibition results plotted as a function of concentration of the scale inhibitor (active) in units of mg/L are shown in FIG. 3. From this data, it is clear that alkyl diphenyloxide sulfonates are also effective agents against calcium sulfate and calcium carbonate scales, with an MIC value of around 560-580 ppm, similar to the MIC value against barium/strontium sulfate scale.

Where a numerical limit or range is stated herein, the endpoints are included. Also, all values and subranges within a numerical limit or range are specifically included as if explicitly written out.

The terms “comprise(s)”, “include(s)”, “having”, “has”, “can”, “contain(s)”, and variants thereof, as used herein, are intended to be open-ended transitional phrases, terms, or words that do not preclude the possibility of additional acts or structures. The present disclosure also contemplates other embodiments “comprising”, “consisting of” and “consisting essentially of”, the embodiments or elements presented herein, whether explicitly set forth or not. As used herein, the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A method of inhibiting the formation of scale in an oil and gas well servicing fluid, the method comprising:
adding a scale inhibitor composition comprising an alkyl diphenyloxide sulfonate into the oil and gas well servicing fluid.

2. The method of claim 1, wherein the alkyl diphenyloxide sulfonate is at least one compound selected from the group consisting of a monoalkyl diphenyloxide monosulfonate, a monoalkyl diphenyloxide disulfonate, a dialkyl diphenyloxide monosulfonate, and a dialkyl diphenyloxide disulfonate.

3. The method of claim 1, wherein the alkyl diphenyloxide sulfonate is a mixture of a monoalkyl diphenyloxide monosulfonate, a monoalkyl diphenyloxide disulfonate, a dialkyl diphenyloxide monosulfonate, and a dialkyl diphenyloxide disulfonate.

4. The method of claim 3, wherein the monoalkyl diphenyloxide monosulfonate and the dialkyl diphenyloxide monosulfonate are present in the mixture in a combined amount of 1 to 15 wt. %, based on a total weight of the mixture.

5. The method of claim 3, wherein the monoalkyl diphenyloxide disulfonate is present in the mixture in an amount of 65 to 93 wt. %, based on a total weight of the mixture.

6. The method of claim 3, wherein the dialkyl diphenyloxide disulfonate is present in the mixture in an amount of 6 to 34 wt. %, based on a total weight of the mixture.

7. The method of claim 3, wherein the monoalkyl diphenyloxide monosulfonate is of formula I, the monoalkyl diphenyloxide disulfonate is of formula II, the dialkyl diphenyloxide monosulfonate is of formula III, and the dialkyl diphenyloxide disulfonate is of formula IV 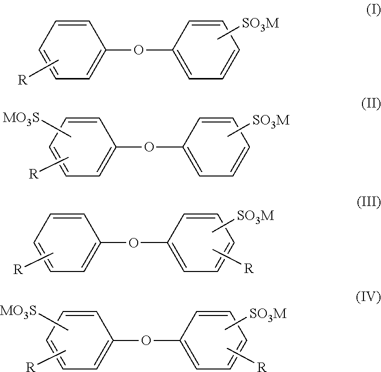
wherein R is an alkyl group with 6 to 22 carbon atoms, and M is selected from H, Na, K, or an ammonium group.

8. The method of claim 7, wherein R is an alkyl group with 9 to 14 carbons, and M is Na.

9. The method of claim 1, wherein the alkyl diphenyloxide sulfonate is added into the oil and gas well servicing fluid at a concentration of 100 to 2,000 ppm.

10. The method of claim 1, wherein the scale inhibitor composition further comprises at least one scale inhibitor selected from the group consisting of a phosphate ester, an organic polymer, a phosphonate, and a carboxylate-containing chelating agent.

11. The method of claim 10, wherein the scale inhibitor composition is a triblend of the alkyl diphenyloxide sulfonate, the phosphate ester, and a sulfonated phosphino polycarboxylic co-polymer.

12. The method of claim 11, wherein a weight ratio of the alkyl diphenyloxide sulfonate to the phosphate ester is 1:3 to 5:1, and a weight ratio of the alkyl diphenyloxide sulfonate to the sulfonated phosphino polycarboxylic co-polymer is 1:3 to 5:1.

13. The method of claim 10, wherein the scale inhibitor composition is a triblend of the alkyl diphenyloxide sulfonate, the phosphate ester, and the phosphonate.

14. The method of claim 13, wherein a weight ratio of the alkyl diphenyloxide sulfonate to the phosphate ester is 1:3 to 5:1, and a weight ratio of the alkyl diphenyloxide sulfonate to the phosphonate is 1:3 to 5:1.

15. The method of claim 11, wherein the triblend is added into the oil and gas well servicing fluid at a concentration of 1 to 10,000 ppm.

16. The method of claim 1, wherein the oil and gas well servicing fluid is formed from produced water or produced water that has been diluted with fresh water.

17. The method of claim 1, wherein the oil and gas well servicing fluid has a total dissolved solids content of 10,000 to 350,000 ppm.

18. The method of claim 1, wherein the oil and gas well servicing fluid comprises ions of sodium, potassium, magnesium, calcium, strontium, barium, chloride, carbonate, bicarbonate, and sulfate.

19. The method of claim 1, wherein the oil and gas well servicing fluid comprises 100 to 10,000 ppm of Ba²⁺ and the scale comprises barium sulfate scale.

20. The method of claim 1, wherein the oil and gas well servicing fluid comprises 100 to 5,000 ppm of Sr²⁺ and the scale comprises strontium sulfate scale.

21. The method of claim 1, wherein the scale inhibitor composition inhibits the formation of scale at temperatures up to 160° C. in the oil and gas well servicing fluid.

22. The method of claim 1, wherein the scale inhibitor composition inhibits the formation of scale at pressures up to 1,000 psi in the oil and gas well servicing fluid.
